(19) United States
(12) Patent Application Publication
(10) Pub. No.: US 2011/0059023 A1
(43) Pub. Date: Mar. 10, 2011

(54) NARROWBAND IMAGING USING NEAR-INFRARED ABSORBING NANOPARTICLES

(76) Inventors: James W. Tunnell, Austin, TX (US); Jon A. Schwartz, Sugar Land, TX (US); Jaesook Park, Seoul (KR); Priyaveena Puvanakrishnan, Austin, TX (US); J. Donald Payne, Houston, TX (US)

(21) Appl. No.: 12/933,145

(22) PCT Filed: Mar. 19, 2009
(86) PCT No.: PCT/US2009/001736

§ 371 Date: Nov. 4, 2010

Related U.S. Application Data
(60) Provisional application No. 61/069,904, filed on Mar. 19, 2008.
(60) Provisional application No. 61/201,627, filed on Dec. 12, 2008.

(51) Int. Cl. A61K 49/00; C12M 1/34; G01N 21/00; A61P 35/00; A61B 6/00

(52) U.S. Cl. 424/9.6; 424/9.1; 435/288.7; 436/164; 600/431

(57) ABSTRACT

Devices and methods are provided for image identification of contrast agents comprising nanopartiles. The imaging devices and methods may also be configured to identify target tissues from surrounding tissues such as disease conditions (e.g. cancer tumors) through narrow-band imaging of tissue. Contrast agents such as near-infrared absorbing nanoparticles may be introduced to an organism to distinguish target cells, tissues, or structures from normal tissue. The contrast agent is allowed to preferentially accumulate in the target cells, tissues or structures. As the contrast agents are adapted to preferentially absorb one or more wavelengths of light, the reflection of one wavelength or band of wavelengths by the contrast agents is compared to a separate wavelength or band of wavelengths to identify the target cells, tissues, or structures. In this way, the comparisons identify the presence of the preferential accumulation of the contrast agent in the target cells, tissues or structures.

### CROSS-REFERENCE TO RELATED APPLICATION

The patent application claims priority to and the benefit of U.S. Provisional Patent Application Ser. No. 61/069,904, filed Mar. 19, 2008, and U.S. Provisional Patent Application Ser. No. 61/201,627, filed Dec. 12, 2008, both of which are herein incorporated by reference.

### STATEMENT REGARDING FEDERALLY SPONSORED RESEARCH OR DEVELOPMENT

This invention was made in part with Government support under grant no. R01 CAI 32032, awarded by the National Institutes of Health. The Government may have certain rights in this invention.

### BACKGROUND

The present invention generally relates to devices and methods for image identification of contrast agents comprising near-infrared absorbing nanoparticles. The imaging devices and methods may also be configured to identify target tissues or distinguish target tissue from surrounding tissues in disease conditions (e.g. cancer tumors) using narrowband imaging to detect the presence of the target tissue. This invention may be used for the identification of tumors or other diseases, but also useful for other identification applications.

Cancer is the second leading cause of death in the United States, with approximately 1.4 million new occurrences and 565,000 deaths each year (American Cancer Society, Cancer Facts and FIGS. 2008). The NIH has reported the total cost of cancer as approximately $219 billion, of which $89B is the direct medical cost, $18B is the indirect cost of associated morbidity and $112B is the indirect cost of mortality. Accordingly, cancer poses a significant problem and economic cost for both the US and other countries.

The most common methods of treatment of cancer are surgery, ionizing radiation, and chemotherapy. The treatment modality for each patient will depend on many factors, including the type of cancer, location(s) of disease, stage, and health of the patient. Surgery is a desired method of treatment if the tumor is in a location where resection is feasible and has a reasonable likelihood of a positive outcome. A significant challenge of surgery is the resection of the tumor without leaving a positive margin (where “positive” indicates cancer is left behind at the margin of the resection) or where surrounding healthy tissue limits the area of resection. The first problem, positive margins, may occur as a result of local infiltrations of the cancer into surrounding tissue (e.g., a common problem in squamous cell carcinomas of the head and neck), the inexactness of imaging modalities such as computed tomography (CT) or magnetic resonance imaging (MRI) as the tissue shifts during surgery, or the proximity to critical nerves or vessels (e.g., a common occurrence in pancreatic resections). The second problem, the need for preservation of surrounding tissue, is critical in resections of cancers in the brain or central nervous system, prostate cancers, pancreatic cancers, squamous cell cancers next to the carotid artery, and many other cancers.

As a result, two established medical “needs” are more precise methods of surgery and more precise tools for the identification of surgical margins. Robotic tools are growing in acceptance for some surgical applications such as prostate cancer, where preservation of nerve bundles, the urethra or colon is important to reduce morbidity. Robotics, however, would benefit from better imaging tools to define the tumor margins. These efforts to reduce morbidity can in some cases result in residual disease, leading to recurrence.

CT, MRI and ultrasound are common tumor imaging methods that have differing levels of precision, but are difficult and/or expensive to use intra-operatively. The procedure requires a temporary halt in the surgery, and these techniques are not easily used with robotic applications. In general, CT, MRI and ultrasound, particularly with conventional contrast agents, rely on the endogenous differences between tumors and surrounding tissue, such as blood perfusion or vascular permeability. While these differences are often detectable, these imaging techniques are best used when a reasonable resection zone is available to allow for their imprecision.

Other techniques have been investigated using the difference in optical properties of tissues. These methods include optical coherence tomography, diffuse optical spectroscopy, Raman scattering, and fluorescence spectroscopy. While these techniques are useful in many applications, the similarities in healthy and cancerous tissue can result in a relatively high false positive and false negative when used to define a tumor boundary. Optical techniques are also best suited for intra-operative procedures or tumors near the surface because of the limited depth of optical penetration in tissue.

Accordingly, the traditional method of tumor or tumor margin identification—histopathology—remains the most common tool to identify margins. The excised tissue must be processed and examined by a pathologist during or after surgery, and given the size of surgical area or excised section, the examination by the pathologist may involve sampling rather than a complete analysis. The delay in examining sections can prolong the cost and risk of surgery, and can also be imprecise (being dependent on the tissue section analyzed). For example, one surgical technique, Mohs surgery, used in skin cancer requires a surgical pause while the pathologist examines the excised section for clear surgical margins.

Various techniques have been investigated to identify the margins of tumors as an aid to surgery. Optical spectroscopy has been used to distinguish between normal brain tissue from tumor. In one example, relying on the different optical properties of normal and tumor tissue, white light reflectance and 337-nm fluorescent spectroscopy was used to distinguish normal tissue from tumor with a sensitivity of 80% and a specificity of 89%. Infiltrating tumor margins were distinguished from normal tissue with a sensitivity of 94% and a specificity of 93%. This work relied on the optical properties of normal tissue compared to tumors, and the demonstrated level of sensitivity and sensitivity was lower than desired.

Others have used conventional contrast agents for margin identification. In one example, a paramagnetic iron oxide conjugated with a Cy5.5 near-infrared emitting dye was delivered intravenously, allowed to accumulate in the tumor. Thereafter, the animals were sacrificed. Blood was drained from the animals to reduce background (potentially reducing the background caused by blood in the dye-emission wavelength) and the tumors were imaged. The border of the tumor as indicated by a probe correlated well with other measurements of the tumor boundary. In other work, a near-infrared fluorescent small molecule specific for PSMA was used to provide image guidance for surgery. Two challenges for this approach include the specificity and universality of the target (PSMA) and the background resulting from near-infrared fluorescence of blood/hemoglobin.

Quantum dots have also been investigated as a conventional contrast agent using the fluorescent emission properties of these particles. A significant issue for the use of cadmium selenide quantum dots is potential toxicity.

More recently, a targeted bioconjugate of the CY5.5 near-infrared fluorescing dye to a chlorotoxin with an affinity for MMP-2 was used for fluorescent imaging. This group achieved positive results in animal models by detecting the fluorescent emissions from this conjugate. Further study may be required to determine any potential cross-reactivity in humans and the photo-bleaching of the near-infrared (NIR) dye in the operating room light. Additionally, fluorescent techniques are hampered by the natural auto-fluorescence of human blood and tissue.

Narrowband imaging (NBI) is a diagnostic technique clinically available today and used in several imaging applications to image morphology near the surface of tissue. NBI is based on the phenomenon that the depth of light penetration in tissue depends on its wavelength, with longer wavelengths penetrating deeper into tissue. This technique illuminates tissue with a broadband source (e.g., white light) and uses narrowband filters in two or more wavelength bands in the visible spectra to capture reflected light. The different wavelength bands are differentially absorbed or reflected by different tissue components, in particular hemoglobin, and allow the visualization of the vasculature near the surface. Again, this technique commonly relies upon the optical properties of endogenous tissue to distinguish healthy and abnormal tissue. The common wavelength bands utilized are in the blue and green area of the spectra, and some techniques also utilize a band in the red wavelength. Relying on the absorption of each of these bands by hemoglobin, and the different transmission depths of these wavelengths into tissue, allows the visualization of surface or sub-surface vasculature or morphology. The visualization of these components has been useful in diagnosing various diseases such as Barrett's esophagus, bladder cancer, oral cancers, etc. However, NBI relies on differences in the endogenous optical properties of tissue, and is thereby limited in applications and specificity for many disease indications.

Accordingly, current investigations involve the use of either (i) the endogenous optical properties of tumor and normal tissue or (ii) electromagnetic emissions from exogenous dyes or materials. The former have inherent limitations because of the natural similarities between tumor and normal tissue, and the latter have limitations because of biocompatibility and photostability.

Therefore, the need for tumor identification, tumor margin identification, and residual disease identification is currently unmet, and new techniques are desired that address one or more disadvantages of the prior art that are biocompatible, robust, sensitive and specific.

### SUMMARY

The present invention generally relates to devices and methods for image identification of contrast agents comprising near-infrared absorbing nanoparticles. The imaging devices and methods may also be configured to identify target tissues or distinguish target tissue from surrounding tissues in disease conditions (e.g. cancer tumors) using narrowband imaging o detect the presence of the target tissue. This invention may be used for the identification of tumors or other diseases, but also useful for other identification applications.

Certain embodiments of the present invention involve the use of a near-infrared absorbing exogenous material and narrowband-pass filters to distinguish a target tissue from its surrounding tissue. The invention described herein may also be referred to herein as near-infrared narrowband imaging (NIR-NBI). Certain embodiments utilize an infrared-absorbing contrast agent to distinguish a target tissue from its surrounding tissue. In particular, if the target to be identified is a tumor, the methods herein may be used to distinguish the tumor from normal tissue. The optical properties of a specific, biocompatible contrast agent may be used to distinguish the target, such as a tumor, from healthy tissue in real-time, with image capture and display within a few seconds or real-time video. The exogenous contrast agent may be selected from among near-infrared absorbing nanoparticles, nanocapsules containing near-infrared absorbing dyes, nanoparticles with near-infrared-absorbing dyes bound to the surface, a targeted near-infrared absorbing dye, or any near-infrared absorbing substance. The dyes described herein may be optionally encapsulated within the nanoparticle, such as within a liposme or micelle. In some cases, the nanoparticle may be coated with one or more of the dyes described herein or the dye may otherwise be affixed to the nanoparticle as desired. Alternatively, the term nanoparticle as used herein may comprise a near-infrared absorbing dye conjugated with a substance, such as a targeting ligand, to cause the dye to preferentially associate with the target.

The contrast agent comprising nanoparticles may be delivered systemically, deposited on a surface, or directly injected into the diseased area, including the lymphatic system. For example, the nanoparticle may be delivered intravenously and accumulate in a tumor through the enhanced permeability and retention (“EPR”) effect. Alternately, the nanoparticle may be actively targeted by conjugating it with a ligand for an endothelial cell receptor, delivering it intravenously, and allowing it to bind at the target site through the ligand. Alternatively, the nanoparticle may be conjugated with a ligand for a cell surface receptor on a target cell, delivered intravenously, and bind to the target cells through the ligand. Alternatively, the nanoparticle may be conjugated with a ligand for a target cell receptor, deposited on a wound bed or surface, and then unbound nanoparticles may be removed, such as by washing, with only nanoparticles bound to the target cells remaining. Alternatively, the nanoparticle may be conjugated with a ligand for a target cell receptor and injected into the diseased area, either interstitially, within the lymphatic system, intrathecally, or by other methods, with natural fluid movements within tissue removing any unbound nanoparticles.

During an imaging procedure, a low-power light may be used to illuminate the surface or wound bed and the reflected light will be captured in one or more wavelengths—one preferentially absorbed by the nanoparticle and one or more wavelengths that are not preferentially absorbed by the nanoparticle. The relative absorption of these wavelengths will allow the identification of the nanoparticles in the target as compared to tissues and blood, enabling the identification of the tumor. The near-infrared wavelength(s) chosen for certain of the methods herein will allow greater depth of penetration through tissue, allowing the identification of the nanoparticle within target tissue beneath the surface illuminated.

Given the absorption profile of the components of human tissue, including water and hemoglobin, it is preferred in certain embodiments that the nanoparticle be selected or manufactured to absorb wavelengths between about 600 nm and approximately 1200 nm. Preferably, the nanoparticle will preferentially absorb wavelengths between about 620 nm and about 850 nm.

The wavelength(s) preferably not absorbed by the nanoparticle may be used for background subtraction and enhancement of the detection of the nanoparticle at a different wavelength. This second wavelength will preferably be between about 400 nm and about 620 nm and in other embodiments between about 400 nm and about 650 nm. Alternatively, this wavelength may be between about 1100 nm and about 3000 nm. As many cameras are designed to detect light at wavelengths below 1200 nm, lower wavelengths may be preferred in certain embodiments. Additionally, light sources are more common in the lower wavelengths.

Alternatively, one or more additional wavelengths detected may be between about 600 nm and about 1100 nm as long as such wavelength is not preferentially absorbed by the nanoparticle.

It will be recognized by one of ordinary skill that this invention with the benefit of this disclosure that NIR-NBI may be combined with traditional narrowband imaging to provide additional information. In these embodiments, the additional near-infared infrared wavelengths imaged allow identification of the presence of the contrast agent as well as the tissue characteristics captured by the narrowband imaging system.

One example of a method of identifying a presence of a tumor comprises: introducing a contrast agent to an organism wherein the contrast agent comprises a plurality of nanoparticles wherein the contrast agent is adapted to preferentially absorb light at one or more wavelengths wherein the one or more wavelengths is between 620 nm and 1200 nm; allowing the contrast agent to preferentially accumulate in the tumor so as to result in a preferential accumulation; illuminating a tissue of the organism with emitted light using a light source; measuring the reflected light at one or more wavelengths preferentially absorbed by the contrast agent so as to produce a first measurement corresponding to the one or more wavelengths; measuring the reflected light at one or more additional wavelengths not preferentially absorbed by the contrast agent so as to produce a second measurement corresponding to the one or more additional wavelengths; and identifying the presence of a tumor as an area of the image of increased contrast between the first measurement and the second measurement.

One example of a method of identifying a presence of disease condition comprises: (a) introducing a contrast agent to an organism wherein the contrast agent comprises a plurality of nanoparticles wherein the contrast agent is adapted to preferentially absorb light at one or more wavelengths between 620 nm and 1200 nm; (b) allowing the contrast agent to preferentially accumulate in the target so as to result in a preferential accumulation; (c) illuminating a tissue of the organism with emitted light using a light source; (d) measuring the reflected light at the one or more wavelengths preferentially absorbed by the contrast agent so as to produce a first measurement corresponding to the one or more wavelengths; (e) measuring the reflected light at one or more additional wavelengths not preferentially absorbed by the contrast agent so as to produce a second measurement corresponding to the one or more additional wavelengths; (f) comparing the first measurement to the second measurement so as to determine a difference between the first measurement and the second measurement; and (g) identifying a presence of disease condition as any point of the first and second measurements where the difference between the first measurement and the second measurement is greater than a predetermined value.

One example of an image identification device for detecting a margin of a disease condition comprises: a contrast agent comprising a plurality of nanoparticles wherein the contrast agent is adapted to preferentially absorb light at one or more wavelengths and wherein the contrast agent is adapted to be introduced into tissue; a light source adapted to illuminate tissue; a detector wherein the detector is adapted to detect light at the one or more wavelengths preferentially absorbed by the contrast agent and wherein the detector is adapted to produce a first measurement corresponding to the one or more wavelengths; wherein the detector is further adapted to detect light at one or more additional wavelengths not preferentially absorbed by the contrast agent and wherein the detector is adapted to produce a second measurement corresponding to the one or more additional wavelengths; a processor communicatively coupled to the detector wherein the processor is adapted to receive the first measurement and the second measurement; and wherein the processor is further adapted to compare the first measurement to the second measurement so as to generate a comparison that identifies a presence of the contrast agent.

One example of a method of identifying an object comprises: marking an object with a contrast agent wherein the contrast agent comprises a plurality of nanoparticles wherein the contrast agent is adapted to preferentially absorb light at one or more wavelengths; illuminating the object with emitted light using a light source; allowing a first portion of the emitted light to be absorbed by the contrast agent and allowing a second portion of the emitted light to reflect to form a reflected light; detecting a first image of the reflected light corresponding to the one or more wavelengths of the reflected light preferentially absorbed by the contrast agent so as to produce a first image corresponding to the one or more wavelengths; detecting a second image of the reflected light corresponding to one or more additional wavelengths not preferentially absorbed by the contrast agent so as to produce a second image; and identifying the presence of the contrast agent as an area of the image of a contrast between the first image and the second image that exceeds a predetermined value.

The features and advantages of the present invention will be apparent to those skilled in the art. While numerous changes may be made by those skilled in the art, such changes are within the spirit of the invention.

### BRIEF DESCRIPTION OF THE DRAWINGS

A more complete understanding of the present disclosure and advantages thereof may be acquired by referring to the following description taken in conjunction with the accompanying figures, wherein:

FIG. 1A illustrates a schematic view of an imaging device in accordance with one embodiment of the present invention.

FIG. 1B illustrates a schematic view of an imaging device in accordance with another embodiment of the present invention.

FIG. 2 illustrates an UV-VIS extinction spectrum of gold nanoshells.

FIG. 3 illustrates contrast images of hemoglobin (Hb) phantom and gold nanoshells (GNS) in the VIS-NIR region with the gray bands representing the narrowband imaging (NBI) wavelengths bands (i.e. VIS image at 580 nm and NIR image at 810 nm).

FIGS. 4(a)-(b) illustrate matrix images of tissue simulating phantoms. More specifically, FIG. 4(a) illustrates composite NBI images, and FIG. 4(b) illustrates standard color images, where 1X, 2X, 5X and 10X refer to varying gold nanoshell concentrations where X is 1.14×10 EXP9 particles/ml.

FIG. 5 illustrates NBI images of small areas from a tissue phantom matrix to demonstrate NBI image characteristics.

FIG. 6 illustrates contrast images provided by gold nanoshell phantoms to estimate detectable concentration in tissue where the error bars represent the ratio of standard deviation to mean signal intensity for different gold nanoshell concentrations and the black line represents the background noise.

FIGS. 7(a)-(d) illustrate narrow band images of a colon tumor grown in a mouse after gold nanoshells were delivered systemically. More specifically, FIGS. 7(a) illustrates a grayscale VIS image (580 nm). FIGS. 7(b) illustrates a grayscale NIR image (810 nm). FIGS. 7 (c) illustrates a composite NBI image. FIGS. 7(d) illustrates a standard color image. FIGS. 7(e)-(h) illustrate narrow band images of control colon tumor. More specifically, FIG. 7(e) illustrates a grayscale VIS image (580 nm). FIG. 7(f) illustrates a grayscale NIR image (810 nm). FIG. 7(g) illustrates a composite NBI image. FIGS. 7(d) and 7(h) illustrate black and white representations of the standard color image.

FIGS. 8(a)-(c) illustrate narrow band images of a human colon tumor grown in a mouse after systemic infusion with gold nanoshells. More specifically, FIG. 8(a) illustrates a grayscale VIS image (580 nm). FIG. 8(b) illustrates a grayscale NIR image (810 nm). FIG. 8(c) illustrates a composite NBI image. The black arrows indicate gold nanoshells in the tumor.

FIGS. 8(d)-(f) illustrate narrow band images of a control tumor (injected with trehalose). More specifically, FIG. 8(d) illustrates a grayscale VIS image (580 nm). FIG. 8(e) illustrates a grayscale NIR image (810 nm). FIG. 8(f) illustrates a composite NBI image.

FIG. 9 illustrates composite NBI images of human colon tumors illustrating heterogeneous distribution of gold nanoshells where the black arrows indicate gold nanoshells in a tumor.

Although the graphs and photos shown herein are shown in grayscale because color images are not available in an International Application, it is explicitly recognized that the grayscale images are capable of being rendered in color as described herein.

While the present invention is susceptible to various modifications and alternative forms, specific exemplary embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DESCRIPTION OF PREFERRED EMBODIMENTS

The present invention generally relates to devices and methods for image identification of contrast agents comprising near-infrared absorbing nanoparticles. The imaging devices and methods may also be configured to identify target tissues or structures or distinguish target tissue from surrounding tissues in disease conditions (e.g. cancer tumors) using narrowband imaging to detect the presence of the target tissue. This invention may be used for the identification of tumors or other diseases, but also useful for other identification applications.

More particularly, the present invention involves the use of near-infrared absorbing contrast agents such as near-infrared absorbing nanoparticles to distinguish target cells, tissues, or structures from normal tissue. The absorption of one wavelength or band of wavelengths by the contrast agents is compared to a separate wavelength or band of wavelengths to identify the target cells, tissues, or structures.

In certain embodiments, methods and devices for identifying the presence of a target tissue from surrounding tissue are provided. Where the target tissue is a disease condition such as a cancer tumor, embodiments of the methods herein comprise introducing a contrast agent to an organism, allowing the contrast agent to preferentially accumulate in the tumor so as to result in a preferential accumulation, and imaging a tissue of the organism so as to identify the presence of the preferential accumulation of the contrast agent in the tissue. The contrast agent may comprise a plurality of nanoparticles and is adapted to preferentially absorb light at one or more wavelengths.

The imaging may be accomplished by illuminating the tissue of the organism with emitted light using a light source. If the near-infrared absorbing contrast agent is not present in the tissue, wavelengths in the near-infrared may be reflected by the tissue and the intensity of such reflected light may be detected. If the near-infrared absorbing contrast agents are present in the tissue, a portion of the illuminating light will be absorbed by the contrast agent and less of the illuminated light will be reflected. A detector may used to identify one or more regions of the area illuminated where the intensity of the illuminated light is lower, indicating the presence of the nanoparticles. A second or more wavelengths or band of wavelengths may also be detected to provide a baseline for measurement, these wavelengths being ones not preferentially absorbed by the nanoparticle. The intensity of the reflected light in these wavelengths may be detected and used to provide a baseline for the wavelengths used to identify the presence of the contrast agent, including being used in algorithms to determine sensitivity.

These detected wavelengths may be processed into images, and color-coded in visible wavelengths for display in cameras or monitors during a surgical procedure. In this way, a first image of the reflected light corresponding to the one or more wavelengths of the reflected light may be displayed, which corresponds to the wavelength(s) preferentially absorbed by the contrast agent. A second image of the reflected light corresponding to one or more additional wavelengths not preferentially absorbed by the contrast agent may then be displayed. As used herein, the term “not preferentially absorbed” means absorbed less than the absorption of the one or more wavelengths that are preferentially absorbed by the contrast agent.

By comparing or combining the first and second images, an identification of the presence of the contrast agent in the tissue may be determined as an area of the image of increased contrast between the first image and the second image. In this way, the presence of a tumor and hence, a tumor margin may be identified. Additionally, this imaging technique may be applied to identify the presence of residual disease, as after tumor resection.

Advantages of certain embodiments of the present invention include, but are not limited to, enhanced identification of disease conditions such as cancer tumors, real-time imaging capabilities of disease conditions, more accurate and precise identifications of tumor margins, and enhanced identification of objects marked with nanoparticle contrast agents.

Although certain embodiments are discussed herein in the context of disease condition identification (e.g. identification of cancer tumors), other applications are explicitly contemplated for the methods and devices described herein. Examples of other suitable uses include, but are not limited to, identification of objects marked with a contrast agent affixed to the object. Although any object may be marked with the contrast agents described herein for identification purposes, exemplary objects include, but are not limited to, high-security identification cards, transport vehicles, tanks, financial instruments (e.g. cashier's checks, money orders, various forms of currency), containers, packaging, and other objects that would benefit from the contrast agent marking/identification methods described herein. Additionally, this invention may also be applied to other medical or industrial applications, such as cardiovascular imaging.

To facilitate a better understanding of the present invention, the following examples of certain embodiments are given. In no way should the following examples be read to limit, or define, the scope of the invention.

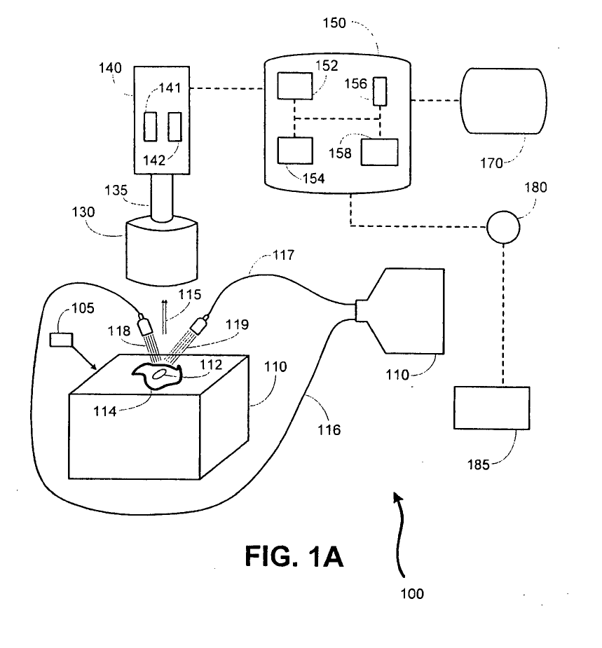

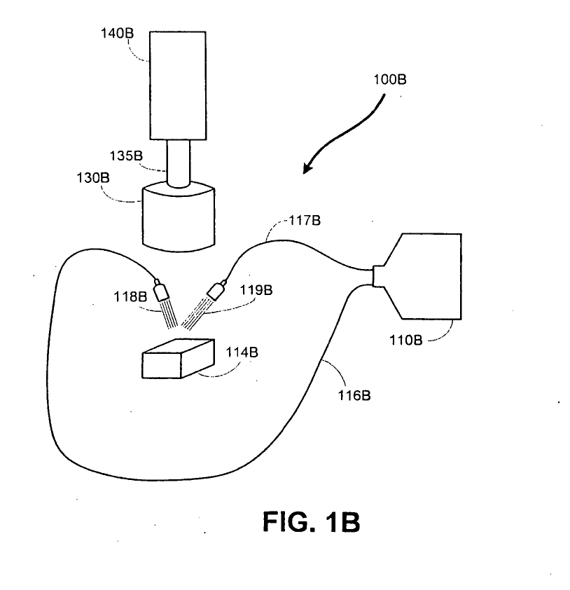

FIG. 1A illustrates a schematic view of an imaging device 100 in accordance with one embodiment of the present invention. Organism 110 is shown schematically in

FIG. 1A, having a tissue 112. A disease condition, in this case, cancer tumor 114 is shown in tissue 112. Light source 110 provides emitted light 118 and 119 to illuminate tissue 112 via fiber optic cables 116 and 117. Emitted light 118 comprises one or more wavelengths of light, and emitted light 119 comprises one or more additional wavelengths of light.

Light source 110 may be any source for emitting light or electromagnetic radiation for illuminating tissue 112. Suitable examples of light source 110 include, but are not limited to, low-power visible light lamps, quartz-tungsten-halogen (QTH) lamps, or any combination thereof. Light source 110 may be a low power lamp. In certain embodiments, emitted light is a broadband visible spectrum. In other embodiments, emitted light 118 and 119 each comprise one or more wavelengths of electromagnetic radiation. Although the emitted light shown in this example is shown as emanating from two separate fiber optic cables, it is recognized that such emitted light may be formed directly from light source 110 directly illuminating tissue 112 without the use of fibers optic cables 116 and 117. Optic cables 116 and 117 may also employ filters to provide illumination of specific wavelengths from each fiber optic cable. The illumination from such cables may also be alternated to provide enhanced illumination and detection.

A contrast agent may be introduced to organism 110 to enhance identification of tumor 114. As explained in further detail below, contrast agent 105 may be comprised of nanoparticles that preferentially absorb light at one or more wavelengths. Contrast agent 105 may be introduced to organism 110 so as to result in a preferential accumulation of contrast agent 105 in tumor 114. Contrast agent 105 may preferentially accumulate in tumor 114 by a variety of mechanisms, either passive or active, which are described below in further detail.

Contrast agent 105 may be systematically introduced to organism 110 as by introduction of contrast agent 105 into the circulatory system (not shown) of organism 110, or contrast agent 105 may be directly deposited on or in tissue 112 as desired such as by topical administration.

Some of emitted light 118 and 119 is absorbed by tissue 112, tumor 114, and contrast agent 105. Some of emitted light 118 and 119 is reflected back as reflected light 115.

Optional filter 130 filters reflected light 115, limiting detection of reflected light 115 to one or more wavelengths as desired. In certain embodiments, filter 130 may be a liquid crystal tunable filter (LCTF), a rotating wheel filter, or any filter known in the art for suitable to filter one or more wavelengths of light.

Optional lens 135 focuses filtered light from filter 130 onto detector 140. Detector 140, is adapted to detect the one or more wavelengths of light preferentially absorbed by contrast agent 105 to form a first intensity measurement or image, and detector 140 is further adapted to detect one or more additional wavelengths of light not preferentially absorbed by contrast agent 105 to form a second intensity measurement or image. By comparing the first measurement to the second measurement through the use of information handling system 150, one can identify areas of contrast which correspond to the presence of contrast agent 105. Thus, where contrast agent 105 has preferentially located in tumor 114, this comparison identifies the presence of tumor 114 in tissue 112 as an area of enhanced contrast between the first image and the second image. In certain embodiments, the enhanced contrast will be identified by a difference between the second measurement and the first measurement greater than a predetermined value. In other embodiments, the enhanced contrast is identified by a ratio the second measurement of the first measurement greater than a predetermined value. The predetermined value may be about 20% or in certain embodiments, from about 10% to about 40%, or any value suitable to identify the presence of contrast agents from a comparison of the two measurements.

It is recognized that the first measurement of the one or more wavelengths may occur simultaneously or sequentially with the second measurement of the one or more additional wavelengths as desired.

Often, illumination and detection by broadband visible light alone is insufficient to clearly indicate the location and margins of tumor 114 in tissue 112. Accordingly, the imaging described herein provides an enhanced contrast view showing the presence of the contrast agent. Where the contrast agent has preferentially associated with a target cells, structure, or tissue, this imaging identifies the presence or demarcation of the target cells, structure or tissue.

Although FIG. 1A shows one example of a hardware implementation of imaging device 100, other hardware implementations may be utilized to achieve the same functions noted herein as would be recognized by a person of ordinary skill in the art with the benefit of this disclosure. For example, in certain embodiments, detector 140 comprises a plurality of detectors, such as first detector 141 and second detector 142. In certain embodiments, the functions of filter 130 and lens 135 are incorporated directly into detector 140 so as to form a single integral detector. Detector 140 may be any device suitable for detecting one or more wavelengths of electromagnetic radiation, including one or more wavelengths of light. Suitable examples of detectors for use with the present invention include, but are not limited to, charge-coupled devices (CCD), analog image detectors, or any combination thereof.

Information handling system 150 comprises microprocessor or CPU 154, memory 156, storage device 158, and video processor 152. Information handling system is shown here as communicatively coupled to display 170 and input/output bus 180. The first and second images may be outputted to or otherwise displayed on display 170 as a combined image or separately as desired. Additionally or alternatively, the images or comparisons thereof may be stored in storage device 158 or in memory 156, which in some embodiments may comprise flash memory. Images and measurements from detector 140 may also be communicated to input/output bus 180 for communication to secondary devices 185. Secondary devices suitable for use with the present invention include, but are not limited to, robotic devices for excising tumors, and/or devices for providing photothermal therapy which, use as one of their inputs, output data from information handling system 150. In this way, secondary devices 185 may be guided by or otherwise receive feedback from information handling system 150.

In certain embodiments, the images and/or comparisons thereof produced are produced a plurality of times and at a rate sufficient to provide real-time video, which may be used during surgery or other medical procedures as desired. In such a procedure, a tumor or other target cells may be removed during the identification methods described herein. In other embodiments, snap-shot imaging is preferred.

In additional embodiments, image device 100 may be adapted for use in endoscopic surgery. More particularly, detector 140 may further comprise one or more fiber optic cables for use in endoscopic procedures along with the optional fiber optic cables of light source 110. Alternately, the device may be a probe for percutaneous use.

Suitable Contrast Agents

Contrast agents suitable for use with the present invention comprise nanoparticles. The term “nanoparticles” as used herein refer to any material adapted to preferentially absorb the desired wavelengths of electromagnetic radiation. To serve as a suitable contrast agent, any material that absorbs strongly in the near-infrared region of the spectrum could also be used. Examples of these materials and their methods of production and functionalization are known in the art. See e.g., U.S. Pat. Nos. 6,344,272 and 6,685,986, which are incorporated by reference. These near-infrared absorbing materials include, among others: nanoshells (including gold-shell silica core nanoshells, gold-gold sulfide nanoshells, hollow nanoshells and other variants), metal nanorods, nanostars, hollow nanoparticles, nanocages, elliptical “nanorice,” carbon particles, buckeyballs, carbon fullerenes, nanocubes, carbon nanotubes, and near-infrared absorbing dyes such as indocyanine green, either conjugated to a targeting ligand or bound to the surface of or contained within another particle.

In certain embodiments, more than one type of nanoparticle may be simultaneously used. Each type of nanoparticle may be designed or tuned to preferentially absorb a different wavelength of external energy. Where the nanoparticle is a nanoshell, for instance, this tuning may be accomplished by changing the ratio of the core to shell thickness. The different types of contrast agents may be referred to as a first contrast agent, a second contrast agent, a third contrast agent, and so on. Where multiple contrast agents are used with each contrast agent tuned to preferentially absorb a different wavelength or range of wavelengths, additional measurements or imaging may be performed to detect these additional wavelengths preferentially absorbed by each additional contrast agent. In the case where a second contrast agent is present, a third measurement would be necessitated to measure the wavelength(s) preferentially absorbed by the second contrast agent.

These nanoparticles may be delivered to the target area, such as a tumor, by injection or by systemic delivery. As described further below, these particles may optionally be targeted to the vasculature associated with the tumor. As used herein, the term “nanoparticle” also includes particles of a size that may be systemically delivered to the target area through the blood stream or lymphatic channels. In certain embodiments, a nanoparticle will have a largest dimension of less than about 1 micron, and in other embodiments, less than about 200 nanometers.

Given the absorption profile of the components of human tissue, including water and hemoglobin, in certain embodiments, nanoparticles may be selected or manufactured to absorb wavelengths between about 600 nm and about 1200 nm, between about 600 nm and about 1100 nm in other embodiments, and between about 700 nm and about 900 nm in still other embodiments. In certain preferred embodiments, the nanoparticles will be designed to preferentially absorb wavelengths between about 620 nm and about 850 nm.

The wavelength(s) preferably not absorbed by the nanoparticle may be used for background subtraction and enhancement of the detection of the nanoparticle at a different wavelength. In certain embodiments, this second or additional wavelength(s) will preferably be between about 400 nm and about 650 nm. Alternatively, this wavelength may be between about 1100 nm and about 3000 nm. Alternatively, one or more additional wavelengths detected may be between 600 nm and 1100 nm as long as such wavelength is not preferentially absorbed by the nanoparticles. Indeed, any wavelength may be chosen for the second or additional wavelength(s) so long as this second or additional wavelength does not fall within the peak absorption cross-section of the contrast agent. Many cameras are designed to detect light at wavelengths below 1200 nm. Accordingly, when using such hardware, these lower wavelengths may be preferred. Additionally, light sources are more common in the lower wavelengths.

In certain embodiments, the contrast agents are inert and biocompatible, meaning that their introduction into an organism or tissue causes no substantial adverse health effects.

### Introduction and Preferential Accumulation of Contrast Agents

The contrast agent may be systemically introduced into the organism to be treated. As used herein, the term “systemic introduction” refers to any introduction of nanoparticles that pertains to or affects the organism as a whole such as an introduction of nanoparticles into the circulating blood of an organism. In addition to systemically introduction of the contrast agent (e.g. intravenously, intrathecally, or through the lymphatic system), the contrast agent may also be directly deposited on or in tissue 112 as desired, such as by a topical administration.

As previously described, the mechanism by which the nanoparticles accumulate in the target area may be by a passive mechanism, an active mechanism, or a combination thereof. Passive mechanisms include, but are not limited to, introducing a contrast agent into the circulatory system (not shown) of organism 110 so as to result in an accumulation of the contrast agent in a tumor by the enhanced permeability and retention (EPR) effect. The enhanced permeability and retention (EPR) occurs where leaky tumor vasculature containing wide interendothelial junctions, abundant transendothelial channels, incomplete or absent basement membranes, and dysfunctional lymphatics contribute to passive extravasation of systemically injected macromolecules and nanoparticles into tumors.

Active mechanisms for targeting the tumor site include conjugating nanoparticles with an antibody to a cell surface molecule, such as an anti-EGFr antibody, preferentially expressed by a target cell. These particles may be inserted into the blood, allowed to selectively accumulate in the target area, and selectively bind to cells in the target area which have such molecules present on their cell surface. Alternatively, the nanoparticle may be conjugated to a ligand (such as cyclic RGD) to an endothelial cell marker present in the vasculature of the tumor (such as the integrin alpha v beta 3).

The nanoparticle may be coated with polyethylene glycol or a similar molecule to allow greater circulation time in the blood. A ligand for a molecule on the cell surface of the target cell may be affixed to the nanoparticle or to this coating. Examples of the conjugation of ligands to nanoparticles are known in the art.

Alternatively, the ligand attached to the nanoparticle may result in endocytosis (such as by phagocytosis or pinocytosis) of the material by target cell. The properties of the nanoparticle may also result in the preferential association and endocytosis by the target cells. In certain embodiments, the nanoparticle may be conjugated with a ligand for a tumor cell receptor, deposited on a wound bed or surface, and then unbound nanoparticles may be removed with only nanoparticles bound to the tumor cells remaining.

A variety of ligands may be selected for use to preferentially associate the nanoparticle with the target cells. The attachment of these ligands to exogenous materials has been extensively described in the scientific literature. The choice of ligand is dependent on the target cells. For example, if the target is a tumor cell of epithelial origin, an antibody or antibody fragment to cytokeratin 8, EpCam or other surface molecules may be used. Alternatively, the ligand may be selected for affinity to the HER2 receptor, the EGF receptor, an integrin, a hormonal receptor, or a variety of other surface molecules. One of ordinary skill in the art, with the benefit of this disclosure, will appreciate, that the ligand may be selected from a variety of proteins, peptides, antibodies, antibody fragments, aptamers or other compounds that has a preferential affinity for the target over other tissue components. In certain embodiments, the ligand is selected from the group consisting of: ligands having an affinity for an integrin, ligands having an affinity for a VEGF receptor, and ligands having an affinity for a PSMA.

In this way, with or without active targeting mechanisms, contrast agents comprising nanoparticles may preferentially accumulate in target cells or a tumor. That is, various ligands and/or the EPR effect can enhance the preferential accumulation of nanoparticles.

It is explicitly recognized that any of the elements and features of each of the devices described herein are capable of use with any of the other devices described herein with no limitation. Furthermore, it is explicitly recognized that the steps of the methods herein may be performed in any order except unless explicitly stated otherwise or inherently required otherwise by the particular method.

It is explicitly recognized that one or more methods of the present invention may be implemented via an information handling system. For purposes of this disclosure, an information handling system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, an information handling system may be a personal computer, a network storage device, or any other suitable device and may vary in size, shape, performance, functionality, and price. The information handling system may include random access memory (RAM), one or more processing resources such as a central processing unit (CPU or processor) or hardware or software control logic, ROM, and/or other types of nonvolatile memory. Additional components of the information handling system may include one or more disk drives, one or more network ports for communication with external devices as well as various input and output (I/O) devices, such as a keyboard, a mouse, and a video display. The information handling system may also include one or more buses operable to transmit communications between the various hardware components.

### EXAMPLES

To facilitate a better understanding of the present invention, the following examples of certain aspects of some embodiments are given. In no way should the following examples be read to limit, or define, the scope of the invention.

The following examples provide applications of near-infrared narrowband imaging (NIR-NBI) for imaging tumors using exogenous contrast agents. In particular, gold nanoshells (GNS), one of the class of near-infrared-absorbing materials available, is used as the exogenous contrast agent. A broadband light is used for illuminating the target and imaging select wavelength bands in the visible (VIS) and NIR regions to enhance visualization of hemoglobin and GNS, respectively. The absorption properties of hemoglobin and GNS in the respective VIS and NIR wavelengths are combined to specifically identify the tumor regions. The narrow wavelength bands providing high contrast for hemoglobin and GNS were quantitatively determined using tissue simulating phantoms. This method found the optimum NBI wavelengths in the VIS and NIR to be 540-580 nm and 620-900 nm, respectively. As explained below, ex vivo NIR-NBI of murine tumors accumulated with GNS were then performed using two bands: 580 nm for highlighting blood and 810 nm for highlighting GNS.

### Materials and Methods

NBI system

FIG. 1B illustrates a schematic view of an imaging device in accordance with another embodiment of the present invention. In particular, FIG. 1B shows a schematic of the NBI system. A quartz-tungsten-halogen lamp (110B) (100 W, Newport Stratford Inc., Stratford, Conn.) was provided for white light illumination (400-1100 nm); a liquid crystal tunable filter (LCTF) (130B) (Meadowlark Optics Inc., Frederick, Co.) was provided for wavelength selection; a lens (135B) was provided for focusing the filtered light; and a cooled 12-bit CCD (140B) (CoolSnap, Photometrics, Tucson, Ariz.) was provided to collect reflected light. A bifurcated fiber optic cable (116B and 117B) (Dolan Jenner, Boxborough, Mass.) focused the light directly onto the sample (114B). LCTF 130B is a tunable band pass filter with a full width at half maximum (FWHM) of ˜5 nm tuned to operate in a wavelength range of 400-1100 nm.

### Gold/Silica Nanoshell Fabrication

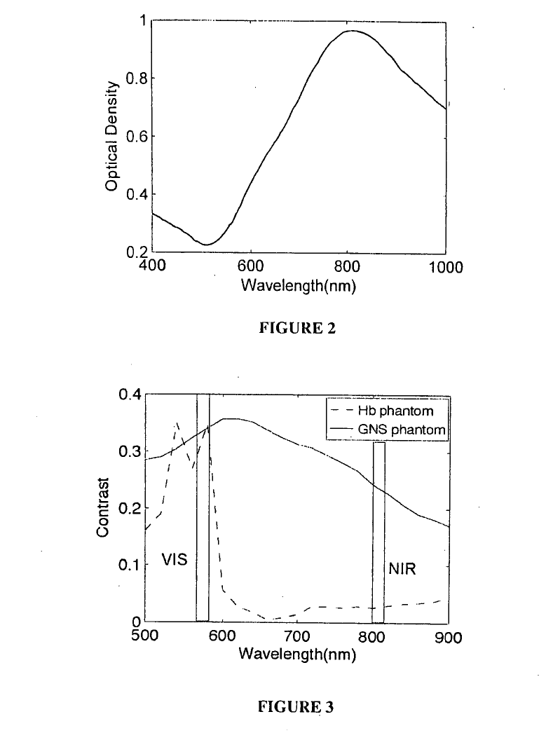

Nanoshells were fabricated is based on the method described in S. J. Oldenburg, R. D. Averitt, S. L. Westcott and N. J. Halas, “Nanoengineering of optical resonances,” CHEMICAL PHYSICS LETTERS 288(2-4), 243-247 (1998). Briefly, gold colloids, 1-3 nm in diameter, were grown over an aminated, 120±12 nm core of colloidal silica (Precision Colloids, LLC, Cartersville, GA). Gold colloid and the particles were then further reacted with HAuCl₄ in the presence of formaldehyde causing the gold surface to grow and coalesce, ultimately forming a complete shell. The gold surface was then pegylated using thiolated polyethylene glycol (SH-PEG) (Laysan Bio, Huntsville, Al.) to improve stability and blood circulation. The finished particles were then suspended in 10% trehalose solution to create an iso-osmotic solution for injection. GNS formation and dispersion in solution were assessed using a UV-Vis spectrophotometer. FIG. 2 illustrates the extinction spectrum of the GNS used in this study. The particles were designed to have a core size of 120 nm and a shell thickness of 15 nm, resulting in an absorption peak between 800 and 810 nm. For passive targeting, a thiolated polyethylene glycol (SH-PEG) (Laysan Bio, Huntsville, Al.) was added to the shell surface by combining 5 μM SH-PEG and GNS in deionized water for 12 hr, followed by diafiltration to remove the excess SH-PEG. The GNS were suspended in an iso-osmotic solution of 10% trehalose solution for injection.

### Tissue Phantoms

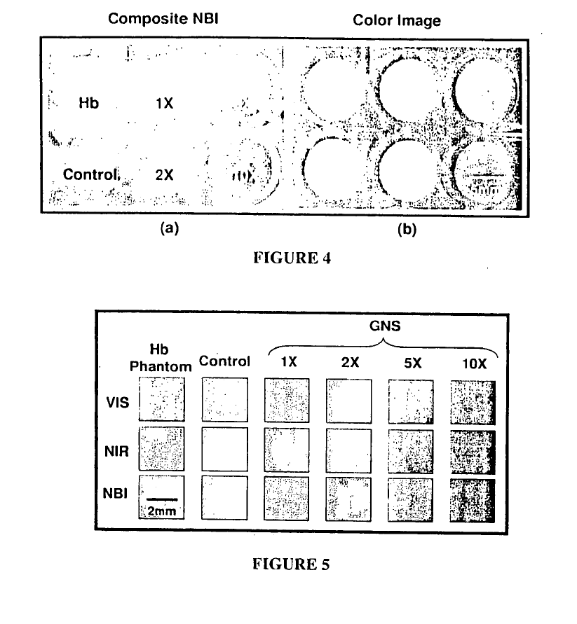

To determine the optimum wavelength bands and the GNS detection limit, tissue simulating phantoms of known optical properties were used. These phantoms were fabricated using polystyrene microspheres (diameter=1.025 μm; Polysciences, Warrington, Pa.) to simulate scattering and hemoglobin (Sigma, St. Louis, Mo.) and GNS to simulate absorption. Mie theory was used to calculate the reduced scattering coefficient (μₛ′(λ) and a spectrophotometer (DU 720, Beckman Coulter, Fullerton, Calif.) to measure the absorption spectra of hemoglobin and GNS . A set of six tissue simulating phantoms was prepared as shown in FIG. 4. Phantom 1 (control phantom) contained polystyrene microspheres in solution (μₛ′(λ₀=630 nm)=1 mm⁻¹), phantom 2 (hemoglobin phantom) contained polystyrene microspheres and hemoglobin ([Hb]=2 mg/ml) and phantoms 3-6 contained varying concentrations of GNS (1X, 2X, 5X and 10X respectively where X refers to 1.14×10⁹ particles/ml concentrations) in polystyrene microsphere solutions. GNS concentration of 1X represents the physiological concentration shown to accumulate in tumors.

### Animal Model

Here, two tumor animal models of colorectal cancer were used to demonstrate the efficacy of NBI. In the first model, Balb/c mice were inoculated with mouse colorectal cancer cells (CT26.WT, ATCC #CRL-2638, mouse colon). Tumors were grown to approximately 5 mm in diameter. The test group (n=3) of Balb/c mice received 4.5 μl/g of GNS solution standardized to an optical density of 100 at 800 nm (2.85×10¹¹ particles/ml) injected intravenously via the tail vein. The control group (n=3) received 4.5 μg saline injections. The mice were sacrificed after 24 hr following GNS injection, and the bulk tissue containing the tumor was excised from the mice in both groups.

In the second model, Swiss nu/nu mice weighing 25-30 g each at four to five weeks old were used. Each animal was inoculated subcutaneously with human colorectal cancer cells (HCT116, ATCC #CCL-247). The test group (n=4) received 4.7 μl/g of GNS solution standardized to an optical density of 97 at 811 nm (2.66×10¹¹ particles/ml) injected intravenously via the tail vein. The control mouse received 4.6 μl/g of the trehalose vehicle.

A second model was used to demonstrate the feasibility of NBI technique to image mice inoculated with human colon cancer cell lines.

### Results

Optimum imaging wavelengths

Here, the optimum imaging wavelengths for NBI using tissue-simulating phantoms was determined. Optimum imaging wavelengths are the wavelengths providing maximum contrast between hemoglobin and GNS in the tumor. Hyperspectral images of the set of tissue-simulating phantoms from the visible to NIR regions (500-900 nm) were collected to determine the optimum imaging wavelengths. An image cube was constructed by collecting intensity images of the phantoms at 22 different wavelengths by tuning the LCTF.

In this study, the contrast was evaluated quantitatively and defined as the luminance ratio (ratio of the difference between sample intensity and background intensity to background intensity) according to Weber's law. In the contrast calculation, the background intensity is that of the control phantom and the sample intensity corresponds to hemoglobin and GNS phantoms. We selected a small region of interest (100×100 pixels) from the imaged sample for each wavelength to calculate contrast for hemoglobin phantom and one GNS phantom (10 X).

A contrast plot for hemoglobin phantom and the GNS phantom in the wavelengths ranging from 500-900 nm is shown in FIG. 3. The hemoglobin phantom contrast peaks at 540 nm and 580 nm, corresponding to the Q-bands of oxy-hemoglobin. The hemoglobin phantom contrast is minimal beyond 620 nm. The GNS phantom's contrast remains high throughout, with the peak at approximately 620 nm. The contrast peak of the GNS phantom appears to have a blue shift relative to the ˜800 nm peak observed in the extinction spectrum of FIG. 2. Therefore, the optimum wavelength bands for enhancing contrast of hemoglobin and GNS are about 540-580 nm and about 620-900 nm, respectively. The subsequent NBI images use two bands: 1) VIS image: 580 nm for highlighting blood and 2) NIR image: 810 nm for highlighting GNS. The gray vertical bands seen in FIG. 3 represent the NBI wavelengths bands.

### NBI of Tissue Phantoms

Narrow band images of tissue simulating phantoms were collected to demonstrate the concept of NBI using the wavelength bands identified in the previous section. Using the standard RGB format, the red channel was assigned to the VIS image and the green channel to the NIR image. The composite NBI image was constructed by overlaying the two images as shown in FIG. 4a. The composite narrow band image visually provides enhanced contrast of hemoglobin phantom and GNS as compared to the standard color image shown in FIG. 4b. Again, although the graphs and photos shown herein in FIGS. 4, 5, 6, 7, 8, and 9 are shown in grayscale because color images are not available in an International Application, it is explicitly recognized that the grayscale images are capable of being rendered in color as described herein.

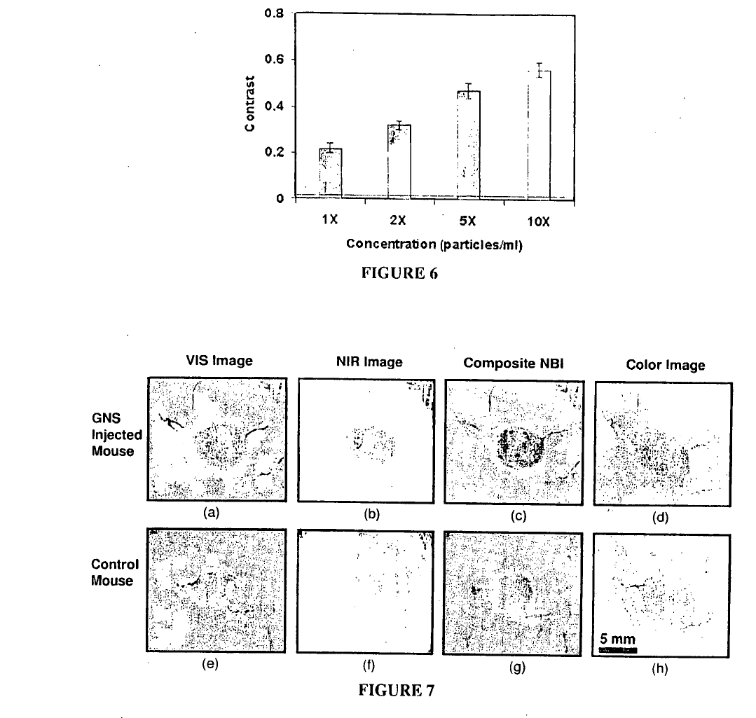

To further demonstrate the NBI image characteristics, small areas were selected from the tissue phantom matrix of VIS and NIR grayscale images and the composite NBI image to present the NBI concept (FIG. 5). A high visual contrast of the hemoglobin phantom is observed in the VIS grayscale image, resulting in a bright red NBI composite image for the hemoglobin phantom. The control phantom has relatively little contrast in either VIS or NIR band resulting in a bright yellow composite NBI image. The GNS phantoms exhibit increasing contrast with higher GNS concentration resulting in an increasingly green NBI image as GNS concentration increased. These tissue phantoms provide an estimate of the detectable concentration of GNS within tissue. The physiological concentration of GNS in tumor (1X) provides at least 20% contrast from the background noise (3%) indicated by the line in FIG. 6. The background noise is the ratio of standard deviation to mean signal intensity of the control phantom. As shown in FIG. 6, the concentration of GNS providing more than 40% contrast is between 5X and 10X.

### NBI of Murine Tumors

An ex-vivo NBI of Balb/c mice inoculated with mouse colon cancer cells after the passive accumulation of GNS was performed. The tumors and their surrounding normal tissue were imaged in the VIS and NIR bands, respectively (FIG. 7). In the ex-vivo VIS images of GNS injected mouse and control mouse, only blood vessels as seen in FIGS. 6a and 6e, respectively were observed. On the other hand, in the NIR images, where the tissue absorption is minimal and the absorption by GNS is maximal, the GNS accumulated tumor regions are clearly defined (FIG. 7b). However, the image of the control tumor in FIG. 7f does not highlight the tumor in the tissue. The composite NBI images of the control and GNS injected tumor are shown in FIGS. 7c and 7g, respectively. As can be seen from the composite NBI image in FIG. 7c, the GNS are highly specific only to the tumor and not to the surrounding tissue. Additionally, the composite narrow band image enhances the contrast provided by GNS accumulated in the tumor compared to the standard color image of the tumor as seen in FIG. 7d.

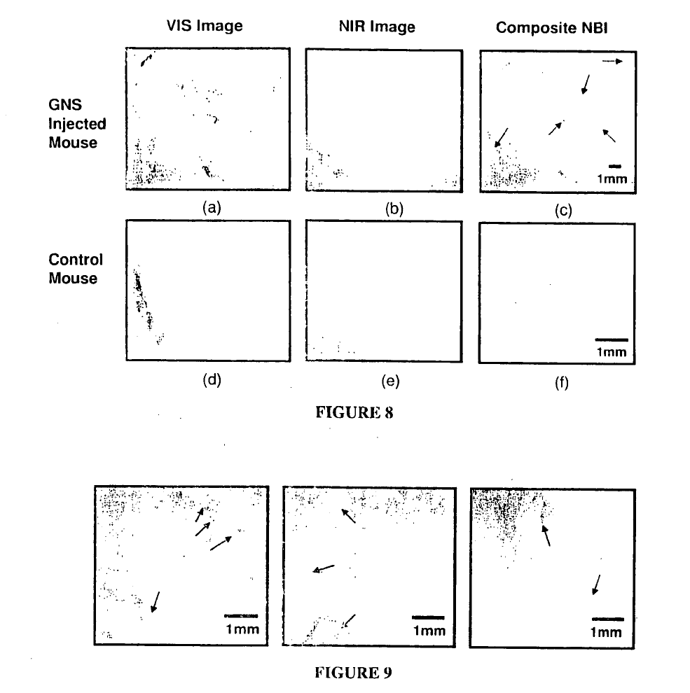

Another set of ex-vivo imaging were performed of Swiss nu/nu mice inoculated with human colon cancer cells. Narrow band images of both the GNS injected mouse and the control mouse were collected. A small portion from the tumor was selected to illustrate the micro distribution of hemoglobin and GNS using the NBI technique. In the ex-vivo NBI images, the blood vessels are clearly visible in the VIS image as seen in FIGS. 8a and 8d. In the NIR image of the GNS injected mouse, the GNS accumulated tumor regions are clearly visible as dark areas as seen in FIG. 8b. The control tumor indicates only the blood vessels as seen in FIG. 8d. The advantage of NBI is clearly demonstrated in the composite NBI images. The composite NBI images of the remaining three mice injected with GNS are shown in FIG. 9 demonstrating the various accumulation patterns seen among the tumors. The black arrows in the images indicate focal regions of higher concentrations of the GNS.

Thus, as illustrated in thess examples, NIR-NBI for imaging GNS systemically delivered to tumors has been demonstrated as an efficacious imaging method that provides enhanced contrast imaging of target cells, structures, and tissues. NBI uses a narrow band of wavelengths matched to the chromophores of interest to highlight contrast between tissue constituents and exogenous contrast agents. Although GNS was used as the NIR absorbing particle to provide contrast between hemoglobin and GNS in tumor, one could use other nanoparticles such as nanorods that can be tuned to absorb in the NIR or organic dyes such as Indo Cyanine Green. In tissue phantoms containing only hemoglobin, a peak contrast was observed between 540 and 580 nm (FIG. 3) consistent with the absorption peaks of hemoglobin. In tissue phantoms containing GNS and no hemoglobin, high contrast was observed throughout the 500-900 nm range with a peak at approximately 620 nm. The GNS with a core diameter of 120 nm and a shell thickness of 15 nm is anticipated to have an absorption peak around 800 nm. While the peak contrast in tissue phantoms is blue shifted from that of pure GNS in solution, the contrast remains high above 600 nm. Therefore, in order to avoid hemoglobin absorption contrast and maintain high contrast from GNS, one should choose a band greater than ˜620 nm. Based on this analysis, hemoglobin contrast can be enhanced by selecting bands between 540-580 nm, and GNS contrast can be enhanced by selecting bands between 620-850 nm. We chose to image tissue phantoms and murine tumors in two bands: VIS image (580 nm) and NIR image (810 nm).

The tissue phantom images obtained using the optimum imaging wavelengths demonstrate the concept of NBI (FIG. 4a). FIG. 5 depicts the intensity variations due to varying concentrations of GNS. In the composite NBI image, the yellow color (control) was observed to correspond to the tissue background. The hemoglobin phantom is assigned a red channel to depict the color of blood in tissue. As the concentration of GNS increase, the intensity decreases due to increase in absorption which is shown by the increasing intensity of green color.

The ex-vivo narrow band images of murine colon tumors indicate that GNS accumulate only in tumors and not in the surrounding normal tissue, suggesting their specificity to tumors. In the magnified ex-vivo images of mice inoculated with human colon cancer cells and injected with GNS presented in FIGS. 8 and 9, GNS's labeled in green are clearly distinguishable from blood vessels labeled in red. The black arrows highlight regions indicating the presence of GNS.

The effects of uneven illumination are the cause of the greenish background in the composite NBI tumor images. In these images, the background should ideally be yellow in color. The punctate areas in these composite NBI images are GNS. Improvement in the target illumination will eliminate shadows and hot spots in the collected images.

The composite NBI image in FIG. 7c demonstrates the specificity of GNS accumulation in the tumor. This demonstrates the potential use of NIR NBI technique for identifying tumor margins pre- and post-resection. Given the high photothermal efficiency of GNS, NBI may be used as a combined imaging and photothermal therapy platform for both identifying and ablating tumors, their margins, and residual disease after resection. Snap shot imaging has been demonstrated in the current study, the simplicity of NBI instrumentation allows for video rate imaging, which could aid in imaging positive tumor margins during surgical resection.

These results show that NIR-NBI can effectively highlight GNS systemically delivered to tumors by illuminating the target using broad band light and collecting narrow band of images in the VIS and NIR to highlight absorption of hemoglobin and GNS. The narrow wavelength bands are quantitatively identified for imaging that provides enhanced visualization of both hemoglobin and GNS in tumors. The results obtained from in vitro and ex vivo imaging show that NIR-NBI is a feasible technique to identify positive margins during surgical resection of tumors. The identification of tumor regions may also be extended for use in image guided surgical removal of tumor margins or photo thermal therapy. The NBI technique may also provide a platform for integrated cancer imaging and therapy.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

What is claimed is:

1. A method of identifying a presence of a tumor comprising:
introducing a contrast agent to an organism wherein the contrast agent comprises a plurality of nanoparticles wherein the contrast agent is adapted to preferentially absorb light at one or more wavelengths wherein the one or more wavelengths is between 620 nm and 1200 nm;
allowing the contrast agent to preferentially accumulate in the tumor so as to result in a preferential accumulation;
illuminating a tissue of the organism with emitted light using a light source;
measuring the reflected light at one or more wavelengths preferentially absorbed by the contrast agent so as to produce a first measurement corresponding to the one or more wavelengths;
measuring the reflected light at one or more additional wavelengths not preferentially absorbed by the contrast agent so as to produce a second measurement corresponding to the one or more additional wavelengths; and
identifying the presence of a tumor as an area of the image of increased contrast between the first measurement and the second measurement.

2. The method of claim 1 wherein the step of allowing the contrast agent to preferentially accumulate comprises allowing the contrast agent to accumulate from an enhanced permeability and retention effect.

3. The method of claim 1 wherein the contrast agent is conjugated with a ligand for a tumor cell receptor so as to enhance the accumulation of the contrast agent in the tumor.

4. The method of claim 1 wherein the contrast agent is conjugated with a ligand for an endothelial cell receptor so as to enhance the accumulation of the contrast agent in the tumor.

5. The method of claim 1 wherein the increased contrast is any contrast greater than about 20%.

6. A method of identifying a presence of disease condition comprising:
(a) introducing a contrast agent to an organism wherein the contrast agent comprises a plurality of nanoparticles wherein the contrast agent is adapted to preferentially absorb light at one or more wavelengths between 620 nm and 1200 nm;
(b) allowing the contrast agent to preferentially accumulate in the target so as to result in a preferential accumulation;
(c) illuminating a tissue of the organism with emitted light using a light source;
(d) measuring the reflected light at the one or more wavelengths preferentially absorbed by the contrast agent so as to produce a first measurement corresponding to the one or more wavelengths;
(e) measuring the reflected light at one or more additional wavelengths not preferentially absorbed by the contrast agent so as to produce a second measurement corresponding to the one or more additional wavelengths;
(f) comparing the first measurement to the second measurement so as to determine a difference between the first measurement and the second measurement; and
(g) identifying a presence of disease condition as any point of the first and second measurements where the difference between the first measurement and the second measurement is greater than a predetermined value.

7. The method of claim 6 wherein the contrast agent is selected to preferentially absorb light between about 620 nm and about 850 nm.

8. The method of claim 6 wherein step (d) further comprises measuring the reflected light at the one or more wavelengths preferentially absorbed by the contrast agent at a plurality of points across the tissue so as to produce the first measurement and wherein step (e) further comprises measuring the reflected light at the one or more additional wavelengths not preferentially absorbed by the contrast agent at the plurality of points across the tissue so as to produce the second measurement.

9. The method of claim 6 wherein step (d) further comprises detecting a first image of an area of the tissue with an image capturing device and wherein step (e) further comprises detecting a second image of the section of the tissue with the image capturing device.

10. The method of claim 9 wherein step (g) further comprises identifying the presence of disease condition as an area of increased contrast between the first image and the second image.

11. The method of claim 10 wherein steps (d) and (e) occur a plurality of times and a rate sufficient to provide a real-time video image of a video wherein the video is a combined image of the first image and the second image.

12. The method of claim 6 wherein the contrast agent is systemically introduced to the tissue of the organism.

13. The method of claim 6 wherein the contrast agent is delivered topically to the tissue of the organism.

14. The method of claim 6 wherein the step of allowing the contrast agent to preferentially accumulate comprises allowing the contrast agent to accumulate from an enhanced permeability and retention effect.

15. The method of claim 6 wherein the disease condition is a tumor having one of: a tumor cell receptor and an endothelial cell receptor, and wherein the contrast agent is conjugated with a ligand selected from the group consisting of: a ligand for the tumor cell receptor and a ligand for the endothelial cell receptor, so as to enhance the accumulation of the contrast agent in the tumor.

16. The method of claim 6 wherein the disease condition is a tumor having a tumor margin wherein step (g) forms an identification of the tumor margin.

17. The method of claim 6 wherein the one or more additional wavelengths not preferentially absorbed by the contrast agent is one or more wavelengths between about 400 nm and about 3000 nm and wherein the one or more additional wavelengths is not within the peak absorption cross-section of the contrast agent.

18. The method of claim 6 wherein the nanoparticle is selected from the group consisting of: metal colloids, carbon particles, nanoshells, nanorods, buckeyballs, carbon fullerenes, carbon nanotubes, nanocages, nanostars, nanocubes, near-infrared absorbing nanoparticles, and any combination thereof.

19. The method of claim 6 wherein the contrast agent further comprises an infrared absorbing dye.

20. The method of claim 19 wherein the infrared absorbing dye comprises indocyanine green.

21. The method of claim 20 wherein an infrared absorbing dye is encapsulated within the contrast agent.

22. The method of claim 6 wherein the disease condition is a tumor and further comprising removing the tumor wherein steps (b)-(g) occur during the step of removing.

23. The method of claim 6 wherein the emitted light comprises electromagnetic radiation at one of the one or more wavelengths preferentially absorbed by the contrast agent and at one of the one or more additional wavelengths not preferentially absorbed by the contrast agent.

24. The method of claim 23 wherein the one or more wavelengths is between about 620 nm and about 900 nm.

25. The method of claim 23 wherein the one or more additional wavelengths is between about 400 nm and about 620 nm

26. The method of claim 23 wherein the one or more additional wavelengths is between about 900 nm and about 3000 nm.

27. The method of claim 6 wherein a ligand to a molecule on the cell surface of a diseased cell is attached to the contrast agent.

28. The method of claim 6 where the disease condition is a cancer.

29. The method of claim 6 wherein step (d) occurs simultaneously with step (e).

30. The method of claim 6 wherein step (d) does not occur simultaneously with step (e).

31. The method of claim 6 wherein the increased difference is any difference beyond a predetermined value.

32. The method of claim 6:
wherein the contrast agent comprises a first contrast agent and a second contrast agent;
wherein the first contrast agent comprises a first plurality of nanoparticles adapted to preferentially light at the one or more wavelengths;
wherein the second contrast agent comprises a second plurality of nanoparticles adapted to preferentially light at a second one or more wavelengths; and
further comprising measuring the reflected light at the second one or more additional wavelengths so as to produce a third measurement corresponding to the second one or more additional wavelengths.

33. The method of claim 6 wherein the step of introducing comprises applying the contrast agent directly to a surface of the tissue.

34. The method of claim 6 wherein the step of introducing comprises injecting the contrast agent directly into the tissue.

35. The method of claim 6 wherein the step of introducing comprises injecting the contrast agent intrathecally.

36. An image identification device for detecting a margin of a disease condition comprising:
a contrast agent comprising a plurality of nanoparticles wherein the contrast agent is adapted to preferentially absorb light at one or more wavelengths and wherein the contrast agent is adapted to be introduced into tissue;
a light source adapted to illuminate tissue;
a detector wherein the detector is adapted to detect light at the one or more wavelengths preferentially absorbed by the contrast agent and wherein the detector is adapted to produce a first measurement corresponding to the one or more wavelengths;
wherein the detector is further adapted to detect light at one or more additional wavelengths not preferentially absorbed by the contrast agent and wherein the detector is adapted to produce a second measurement corresponding to the one or more additional wavelengths;
a processor communicatively coupled to the detector wherein the processor is adapted to receive the first measurement and the second measurement; and
wherein the processor is further adapted to compare the first measurement to the second measurement so as to generate a comparison that identifies a presence of the contrast agent.

37. The image identification device of claim 36 wherein the comparison comprises a difference between the first measurement to the second measurement wherein the presence of the contrast agent is indicated when the difference exceeds a predetermined value.

38. The image identification device of claim 36 wherein the comparison comprises a ratio of the second measurement to the first measurement wherein the presence of the contrast agent is indicated when the ratio exceeds a predetermined value.

39. The image identification device of claim 36 wherein the processor is further adapted to store the comparison in memory and wherein the processor is further adapted to output the comparison to a display.

40. The image identification device of claim 36 further comprising:
a filter adapted to filter light received by the detectors wherein the light source is a quartz-tungsten-halogen lamp;
a lens adapted to focus light from the filter to the detector wherein the filter is a liquid crystal tunable filter wherein the detector is a charge-coupled device; and
wherein the light source further comprises source optical fibers for guiding the emitted light to the tissue and wherein the detector further comprises detector optical fibers for the endoscopic tumor identification.

41. The image identification device of claim 40:
wherein the detector is comprised of a first detector and a second detector;
wherein the first detector is adapted to produce a first measurement corresponding to the one or more wavelengths; and
wherein the second detector is adapted to produce a second measurement corresponding to the one or more additional wavelengths.

42. The image identification device of claim 40:
wherein the first measurement corresponds to a first image of the tissue; and
wherein the second measurement corresponds to a second image of the tissue.

43. A method of identifying an object comprising:
marking an object with a contrast agent wherein the contrast agent comprises a plurality of nanoparticles wherein the contrast agent is adapted to preferentially absorb light at one or more wavelengths;
illuminating the object with emitted light using a light source;
allowing a first portion of the emitted light to be absorbed by the contrast agent and allowing a second portion of the emitted light to reflect to form a reflected light;
detecting a first image of the reflected light corresponding to the one or more wavelengths of the reflected light preferentially absorbed by the contrast agent so as to produce a first image corresponding to the one or more wavelengths;
detecting a second image of the reflected light corresponding to one or more additional wavelengths not preferentially absorbed by the contrast agent so as to produce a second image; and
identifying the presence of the contrast agent as an area of the image of a contrast between the first image and the second image that exceeds a predetermined value.

44. The method of claim 43 wherein the object is a vehicle, an identification card or a financial instrument.

45. The method of claim 44 wherein the step of marking comprises affixing the contrast agent to the object.

46. The method of claim 45 wherein the predetermined value is greater than about 10% to about 40%.
